(19) United States
(12) Patent Application Publication
(10) Pub. No.: US 2020/0163822 A1
(43) Pub. Date: May 28, 2020

(54) DEVICE AND METHOD FOR OPENING AN AIRWAY

(71) Applicant: SOMMETRICS, INC., Vista, CA (US)

(72) Inventor: Jerome K. AARESTAD, Escondido, CA (US)

(73) Assignee: SOMMETRICS, INC., Vista, CA (US)

(21) Appl. No.: 16/636,298

(22) PCT Filed: Aug. 3, 2018
(86) PCT No.: PCT/US2018/045135

§ 371 Date: Feb. 3, 2020

Related U.S. Application Data
(60) Provisional application No. 62/541,065, filed on Aug. 3, 2017.

(51) Int. Cl. A61H 9/00; A61M 1/00; A61F 5/56

(52) U.S. Cl. CPC A61H 9/0057; A61M 1/0088; A61H 2201/1645; A61H 2201/1611; A61H 2201/0192; A61F 5/56

(57) ABSTRACT

The present invention provides devices and methods for creating and/or maintaining patency of the upper airway passage. The device is configured to fit under the chin of a subject at an external location corresponding approximately with the subject's internal soft tissue associated with the neck's anterior triangle. The device includes a flexible self-supporting chamber structure with multi-radius geometry including a particular chamber height range relative to the chamber base span to optimize the chamber's ability to maintain shape when a therapeutic level of negative pressure is applied within the therapy chamber.

The present application claims the benefit of U.S. Provisional Application No. 62/541,065, filed Aug. 3, 2017, which is hereby incorporated by reference in its entirety including all tables, figures, and claims.

### BACKGROUND OF THE INVENTION

The following discussion of the background of the invention is merely provided to aid the reader in understanding the invention and is not admitted to describe or constitute prior art to the present invention.

The external application of negative pressure to patients for palliative or therapeutic purpose is well established in the medical arts.

U.S. Pat. No. 5,343,878, 7,182,082, and 7,762,263 describe various devices which purport to utilize external application of negative pressure upon the external neck surface of patients. A therapeutic appliance is typically provided that has a surface which is configured to enclose an external area of the throat (the term “throat” as used herein referring to the anterior portion of the neck extending approximately from the chin to the top of the sternum and laterally to a point posterior to the external jugular vein) overlying a portion of the upper respiratory passage. In certain embodiments, these appliances can provide a chamber element (e.g., a hollow space filled with air molecules) lying between the interior surface of the chamber element and the throat. The therapy appliance is operably connected to an air pump which is configured to produce a partial negative pressure in this chamber element. Application of a therapeutic level of negative pressure in the chamber element elicits movement of the upper airway and may alleviate conditions such as snoring, sleep apnea, and full or partial airway collapse for example.

In these “negative pressure” therapeutic apparatuses and methods it is difficult to obtain a proper and comfortable fit between the apparatus and the patient to create and maintain the differential negative pressure (relative to atmospheric pressure for example) at the desired location on the patient. This is particularly true as the devices are intended for daily wear for many hours. Success of these negative pressure apparatuses is optimized by a device's ability to accommodate (flex, bend, flow, etc.) varying anatomical features (i.e. device compliance) and accomplished through the use of flexible materials that are formed in structurally resilient geometries to maintain therapeutically effective shapes under a variety of negative pressures.

### BRIEF DESCRIPTION OF THE INVENTION

It is an object of the invention to provide a therapy device with a flexible, self-supporting sealed chamber element that is intended to attach and seal to a patient's external tissue, such as a face, a neck, an area surrounding a wound, etc. This therapy device is particularly suited for forming a sealed chamber element that is configured for the administration of negative pressure to a targeted therapy on the external tissue of an individual.

In a first aspect, the invention provides therapy devices configured for the administration of negative pressure upon the external surface of the individual. These therapy devices comprise:

A self-supporting flexible chamber structure adapted for the administration of negative pressure upon the external surface of an individual comprising:
a) a pressure vessel in the form of an approximately spherocylindrical segment comprising a flexible central region of the pressure vessel defining an arch of a height, chordal span, thickness and composed of a material selected to provide minimal bending stiffness such that the internal volume enclosed by the pressure vessel is maintained under negative pressure over a range of neck angles,
wherein an arch section comprises a height to ½ chordal span length ratio of approximately 0.65-0.85,
wherein a vertical load vector (FAy) is the pressure multiplied by the ½ the chamber span

FAy=P*S,
wherein the vertical load vector is the load vector as a measure from the apex of the chamber element toward the chordal span,
wherein a horizontal load vector (FAx), is the total load vector (FBx) minus the sum of the negative pressure within the chamber (P), multiplied by the chamber height (H)

FAx=FBx−(P*H)
wherein the total load vector (FBx) is defined by the equation:

FBx=P((S²)+(H²))/2H
wherein P is the negative pressure within the chamber, S is the length of ½ the chordal span of the chamber and H is the height of the chamber measured from the top of the arch to the chordal span line,
wherein at the point of contact of the chamber to the skin of the individual, the total load vector angle (α) is approximately between 65 and 79 degrees, a neck to chordal span line angle (β) is approximately between −18 and −24 degrees, the ratio of the total load vector (FBx) to the translated outward load vector ranges from 0 to −0.3 and the inward deflection of the arch is between 0 and 0.4 inches,
wherein the total load vector angle (α) is defined by the equation:

α=arctan (FAy/FAx)
wherein the translated lateral load vector (Fal) is defined by the equation:

Fal=FBx*COS(αβ),
wherein α is the angle of the neck with respect to the chordal span line;
b) an aperture through the self-supporting flexible chamber element; and
c) an air pump operably connected to the chamber element through the aperture to produce a therapeutic level of negative pressure within the chamber element.

The terms “external area” and “external surface” of an individual as used herein refers to a portion of the external skin surface of the individual. In various embodiments, the therapy device is configured to provide optimized fitting parameters, for example, seal, comfort and local device compliance throughout all points of contact. This is preferably achieved by minimizing the contact pressure differential from one point of contact on the skin of a patient to another through design features of the flange element and design features of the sealed chamber element of a negative pressure therapy device.

In various embodiments, the chamber element is affixed to a flange element as an integral structure, as a unitary structure, or as discrete structures.

In certain embodiments, the flange element comprises a tacky material inherent in, or positioned on, all or a portion of the contact area. By way of example only, the tacky material can comprise a room-temperature vulcanizing (RTV) silicone. The tacky material may be a single layer, or may be a component of a lamination stack of materials positioned on all or a portion of the contact area.

In certain embodiments, the flange element is increased in thickness at the junction formed between the flange element and the chamber element, relative to thickness at the edges of the flange element. This thickness may be varied at different points on the flange element. By way of example, at the first and third locations the flange element thickness at the junction formed between the flange element and the chamber element may be between about 0.05 inches and about 0.120 inches, and the flange element thickness at the edge is between about 0.005 inches and about 0.025 inches; while at the second location, the flange element thickness at the junction formed between the flange element and the chamber element is between about 0.05 inches and about 0.20 inches, and the flange element thickness at the edges is between about 0.05 inches and about 0.120 inches; and at the fourth location, the flange element thickness at the junction formed between the flange element and the chamber element is between about 0.020 inches and about 0.100 inches, and the flange element thickness at the edges is between about 0.005 inches and about 0.020 inches.

In preferred embodiments, the flange element has curved profile on the top surface thereof and a flattened profile on the bottom surface thereof. This type of profile provides the increased thickness at the junction formed between the flange element and the chamber element, relative to the edges thereof.

Any and all air pump types find use in the present invention, provided that a therapeutic level of negative pressure can be achieved by the pump. In certain embodiments, the air pump is connected to the apparatus via a hose or tube. Preferably, the air pump is wearable by the patient and is battery powered, and most preferably the air pump is configured integrally to the apparatus. In certain embodiments, the air pump may be a manual squeeze bulb, or may be electric and comprise a piezoelectric material configured to provide an oscillatory pumping motion. It is most preferred that the oscillatory pumping motion operates at a frequency greater than 500 Hz.

In those embodiments where the air pump is configured integrally to the apparatus, the chamber element can comprise an opening into which the air pump engages, wherein when engaged a periphery of the opening forms an airtight seal with the air pump. A compliant sealing ring may be provided within the opening into which the air pump engages. This compliant sealing ring may be provided integrally with the chamber element, and most preferably as a unitary structure with the chamber element. Alternatively, the compliant sealing ring and the chamber element are discrete structures, where the sealing ring may be in the form of a separate O-ring for example. As an alternative to providing the compliant sealing ring as a component of the chamber element, the compliant sealing ring may be provided as a component of the air pump. A compliant sealing ring may also be created via surfaces designed to provide and receive a lip seal. As used herein a lip-type seal consists of a substantially cylindrical compliant flange or tang designed to receive and seal statically against a matching substantially cylindrical surface, for example wherein the lip seal is integrated in to the chamber element and the housing of the air pump forms an air-tight seal when inserted into the chamber element. As used herein, substantially cylindrical refers to a shape that includes but is not limited to a round cylinder an oval cylinder and shapes that lack sharp edges wherein a sharp edge can be defined as a point where two vectors intersect creating a corner-like element.

In certain embodiments, the chamber element comprises one or more apertures creating vent elements that provide an airflow into the chamber when the therapy device is mated to the individual and a therapeutic level of negative pressure is applied. The apertures, located distal to the intake of a pump element provide a flow of air through the chamber that may assist to decrease temperature and humidity within the interior of the chamber. The aperture(s) providing an airflow that is preferably between about 10 mL/min and about 300 mL/min, and most preferably between about 20 mL/min and about 150 mL/min, and still more preferably between about 30 mL/min and about 100 mL/min. Most preferably, the airflow is at least about 40 mL/min or more.

In some embodiments the vent element can comprise an aperture and a filter element within the aperture, wherein the filter element comprises a pore size of about 1.0 μm or less, such as a pore size of about 0.7 μm. The filter element can be configured as a replaceable element and the size adjusted to provide an airflow preferably between about 10 mL/min and about 300 mL/min, and most preferably between about 20 mL/min and about 150 mL/min, and still more preferably between about 30 mL/min and about 100 mL/min.

In yet another embodiment, the vent element can comprise one or a plurality holes distal to the intake of the pump element and of a sufficiently small size to exclude debris from entering the chamber. The hole size further enables the desired airflow of preferably between about 10 mL/min and about 300 mL/min, and most preferably between about 20 mL/min and about 150 mL/min, and still more preferably between about 30 mL/min and about 100 mL/min, wherein the hole size is between about 25 um to about 200 um and more preferably an airflow of at least about 40 mL/min with a hole size between about 70 microns to about 90 microns, and more preferably between about 75 microns to about 85 microns.

Alternatively, the level of airflow can vary. In certain embodiments, the level of airflow tied to the therapeutic level of negative pressure; that is, a higher level of negative pressure can be accompanied by a higher level of airflow due to the differential in pressure between the atmospheric side of the vent elements and the interior of the chamber. In certain embodiments, the negative pressure source may be used in a variable manner to maintain the therapeutic level of negative pressure within a specified range rather than a single value, and the level of airflow can vary in concert with the level of negative pressure.

It is preferred that the chamber element comprises an unloaded spacing measured between the first and third locations that is narrower than a spacing obtained when the chamber element is mated to the individual and the therapeutic level of negative pressure is applied within the chamber element. This unloaded spacing can impart a preload force to the individual by the chamber element prior to the application of negative pressure.

In related aspects, the present invention relates to methods of applying negative pressure therapy to an individual in need thereof, comprising mating a therapy device as described herein to the individual, and applying a therapeutic level of negative pressure within the chamber element, thereby increasing patency of the airway of the individual. Such methods can be for treatment of sleep apnea; for treatment of snoring; for treatment of full or partial upper airway collapse; for treatment of full or partial upper airway obstruction; for negative pressure treatment of a wound caused by, for example an injury or a surgery; etc.

### BRIEF DESCRIPTION OF THE DRAWINGS

FIG. 1 is a side view of an illustrative embodiment of the therapy device including the self-supporting flexible membrane 100, the outer surface of the flange 110, the peripheral edge of the self-supporting flexible membrane 115, an aperture through the chamber 120 and outer surface of the chin-cup element/outer surface of the flange 130.

FIG. 2 is a rear view of an illustrative embodiment of the therapy device including the self-supporting flexible membrane 100, the outer surface of the flange 110, an aperture through the chamber 120, a line bisecting the chamber 125 for purposes in FIG. 3, and inner surface of the chin-cup element/inner surface of the flange element 135.

FIG. 3 is a side view of an approximate half of the device bisected along the line 125 in FIG. 2 including the self-supporting flexible membrane 100, inner surface of the chin-cup of the flange element 135 and dashed lines indicating the measurement points for the height of the chamber 145, chordal span/chordal span line of the chamber 140 and upper spring point 147 and lower spring point 149 of the arch of the chamber.

FIG. 4 is a reoriented side view of an approximate half of the device bisected along the line 125 in FIG. 2 placing the height 145 of the chamber element on the Y axis and the chordal span 140 of the chamber element on the X axis, including the self-supporting flexible membrane 100 an inner surface of the flange corresponding to the chin-cup 135 and dashed lines indicating the measurement points for the height of the chamber 145, chordal span/chordal span line of the chamber 140 and approximately ½ the chordal span/chordal span line of the chamber 140.

FIG. 5A is a partial section view of an approximate half of the device as called out in FIG. 4, 143 with the flange element 137 removed, including a portion of the self-supporting flexible membrane 100, a dashed line indicating the chordal span line 140, solid arrow 160 on the Y-axis showing the approximate vertical load vector component, solid arrow 165 on the X-axis showing the approximate horizontal load vector component, where the X-axis and Y-axis are relative to the chamber height 145 and chordal span line 140.

FIG. 5B is a partial section view of an approximate half of the device as called out in FIG. 4, 143 with the flange element 137 removed, including a portion of the self-supporting flexible membrane 100, a dashed line indicating the chordal span line 140, a solid arrow 150 showing the approximate total load vector as a sum of the vector components in FIG. 5A, 160 and 165, and a curved arrow 155 indicating the total load vector angle (α) with respect to the chordal span line 140.

FIG. 6 is a section view of an approximate half of the device bisected along the line 125 in FIG. 2 including a portion self-supporting flexible membrane 100, a dashed line indicating chordal span line 140, solid arrow 160 showing the approximate perpendicular load vector, a solid arrow 165 showing the approximate lateral load vector, dashed line 170 showing a line parallel to and corresponding to the approximate contact surface of the individual for purposes of measuring the neck angle (β) 175 and a solid arched arrow 175 showing the approximate neck angle (β) as measure between the chordal span line 140 and the line parallel to the contact surface of the individual 170. The P-axis and L-axis markings indicate the directions of the perpendicular and lateral force vectors relative to the surface of the skin respectively.

FIG. 7 is a graphical representation of the tendency for the chamber edge to deflect at a location approximately corresponding to the chamber edge/spring points vs. the angle of the neck (β) wherein the angle of the neck is measured at the point where the chamber edge makes contact with the skin of the user. The ratio of the lateral load vector to the total load vector is on the Y-axis and the neck angle (β) as measured relative to the chordal span line in degrees is on the X-axis. The shaded box depicts parameters where the tendency for the chamber edge to deflect is outward and the unshaded area depicts parameters where the tendency for the chamber edge to deflect is inward.

FIG. 8 depicts a region approximately corresponding to the thyroid cartilage bounded by the dotted lines 180, a region approximately corresponding to gonion bounded by the dotted line 185 and a region approximately corresponding to the mental protuberance bounded by the dotted lines 190.

FIG. 9 depicts a 2-dimentional illustration of a spherocylinder 200 with dashed lines separating the center cylinder segment 205 and spherical end segments 210.

FIG. 10 depicts a 2-dimentional illustration spherocylinder 200 with a dashed line bisecting its longitudinal axis 215 for purposes in FIG. 11.

FIG. 11 depicts a 2-dimentional illustration of an approximate half of a spherocylinder as bisected in FIG. 10, 215 with dashed lines separating the center approximate half cylinder segment 205 and approximate quarter spherical end segments 210.

FIG. 12 depicts a three-dimensional view of an approximate half of a spherocylinder 200 as bisected in FIG. 10, 215 comprising a concave curvature perpendicular to the longitudinal axis of the chamber, an approximate half of a cylinder segment 205, approximate quarter spherical end segments 210 and 220 indicating the interior of the chamber.

### DETAILED DESCRIPTION OF THE INVENTION

The present invention and the various features and advantageous details thereof are explained more fully with reference to the non-limiting embodiments that are illustrated in the accompanying drawings and detailed in the following description. It should be noted that the features illustrated in the drawings are not necessarily drawn to scale. Descriptions of well-known components and processing techniques are omitted so as to not unnecessarily obscure the present invention. The examples used herein are intended merely to facilitate an understanding of ways in which the invention may be practiced and to further enable those of skill in the art to practice the invention. Accordingly, the examples should not be construed as limiting the scope of the invention. In the drawings, like reference numerals designate corresponding parts throughout the several views.

In the present invention, a negative pressure therapy device is designed to maximize the comfort and seal efficiency ultimately optimizing device efficacy and user compliance. The negative pressure therapy device is described below for use in the opening of the upper airway when placed upon the neck of a subject over a surface corresponding to approximately the upper airway of the subject. This exemplary application of the technology is not meant to be limiting. The therapy device comprised of a chamber element and a flange element configured to be the contacting surface between the chamber element and the user described herein is configured to provide for regional load equalization over the interface between a negative pressure therapy device and the three dimensionally varying skin surface of the user so as to maintain a near uniform contact pressure over this non-uniform surface.

In particular, the therapy device referred to herein relates but is not limited to an external therapy appliance for relieving upper airway obstruction. U.S. patent application Ser. No. 12/002,515, 12/993,311, 13/881,836 and 15/360,419 which are hereby incorporated by reference in their entirety including all tables, figures and claims, describes a therapy appliance for relieving airway obstruction. Increasing the patency of the upper airway of an individual alleviates conditions such a snoring, sleep apnea, full or partial upper airway collapse. As described therein, a device is configured to fit under the chin of a user at an external location corresponding to the soft tissues overlying the upper respiratory passages of the neck.

For purposes of the patent application, the term “about” refers to +/− 10% of any given value.

The therapy device of the present invention comprises at least a chamber element and may further comprise a flange element attached to the edge of the chamber element along the circumferential dimension of the flange element to form an airtight junction between the flange element and the chamber element. The junction between the flange element and the chamber element is referred to herein as the “root” of the junction. The location of this root on the flange element may be varied around the circumferential dimension of the flange element for purposes of contact pressure balancing.

As used herein, the term “circumferential dimension” refers to a continuous location along the width of the flange element, in some cases, for example where the chamber element makes continuous contact with the flange element. As used herein, the “root” is the location at which the chamber element contacts the flange element and is of a width enclosed by the thickness of the chamber element. The chamber element may be affixed to the flange element as an integral structure, unitary structure or discreet structures. An “integral structure” refers to a structure that is a complete piece formed by joining two or more components which, once joined, become a single piece that is not separable without destroying the device. A “unitary structure” refers to a structure that is a singular structure formed or molded as a single piece. Two elements are “discrete structures” if the two (or more) structures form a single working structure, but retain individual characteristics and can be separated in the normal course of use of the single working structure and then reassembled.

Surface variation of the therapy site, both permanent and occasional (i.e., the shape of the mandible, transition points from neck to mandible, tissue types, scars, facial hair and/or skin blemishes differential forces applied to different portions of the seal caused by movement of the wearer, etc.) can undesirably disrupt the seal between the negative pressure therapy device and user. The present invention may provide devices, systems and methods of use that can accommodate varying facial contours/features and adapt to movement, resulting in greater comfort, reduced negative pressure leakage and improved therapeutic efficacy.

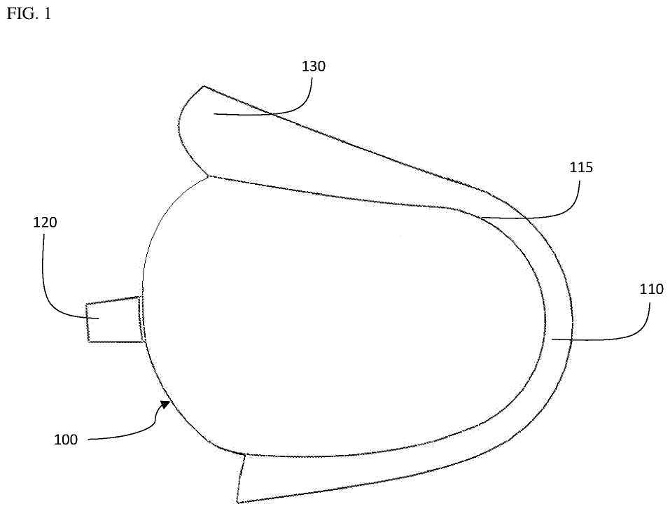

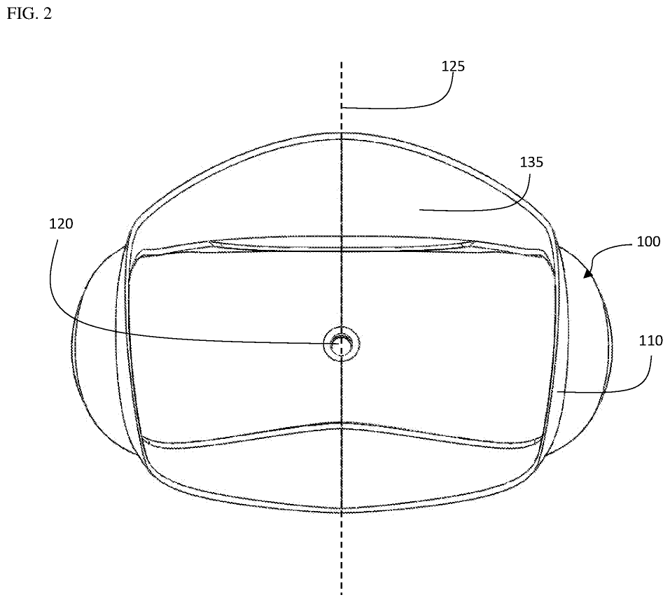

The therapy device of the present invention comprises structural member(s) that interfaces outside a targeted therapy area of a patient. In a preferred embodiment, the therapy area is that of the upper airway. The therapy device contains a chamber element FIG. 1, 100 that is used to create a negative pressure between an inner surface of the appliance and the skin of the upper neck/chin region. The chamber element 100 may be secured to a flange element 110 at a single point along the peripheral edge of the chamber 115 to the back of the flange element 110 that evenly distributes the force across all the flange element. The flange element 110 may further contain features for the positioning, orienting, fitting and or locating of the therapy device on the user. By way of example, FIG. 1, shows the external surface of a chin cup 130. The chamber element 100 may also have an aperture 120 for the insertion of a negative pressure source. The device may be formed, molded, or fabricated from any suitable material or combination of materials. Non-limiting examples of such materials suitable for constructing the therapy appliance include plastics, metals, natural fabrics, synthetic fabrics, and the like. The device may also be constructed from a material having resilient memory such as, but not limited to, silicone, rubber, or urethane.

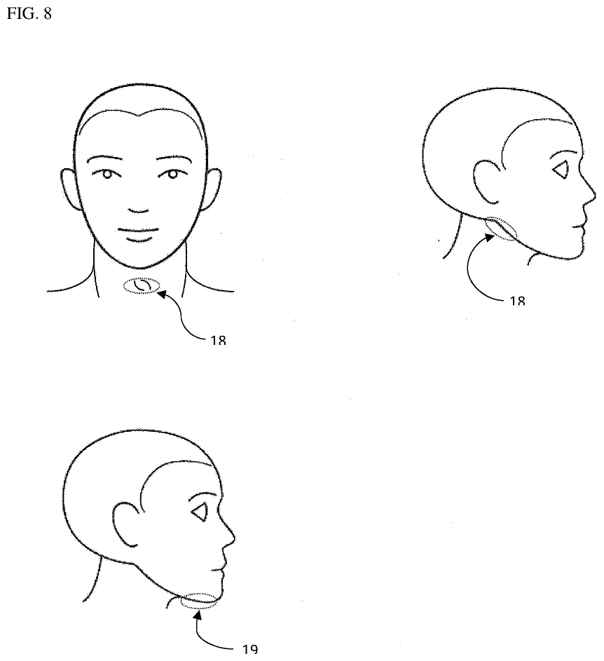

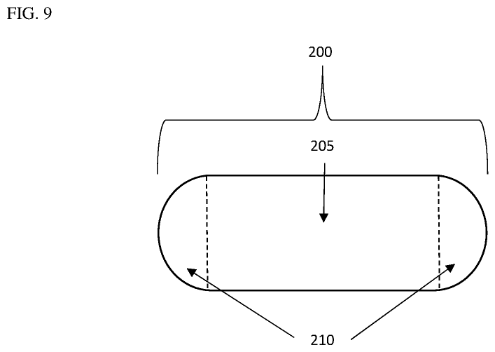

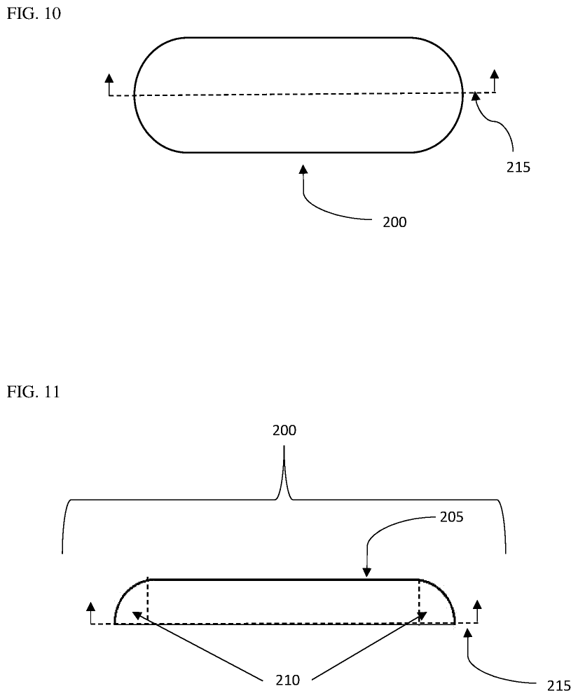

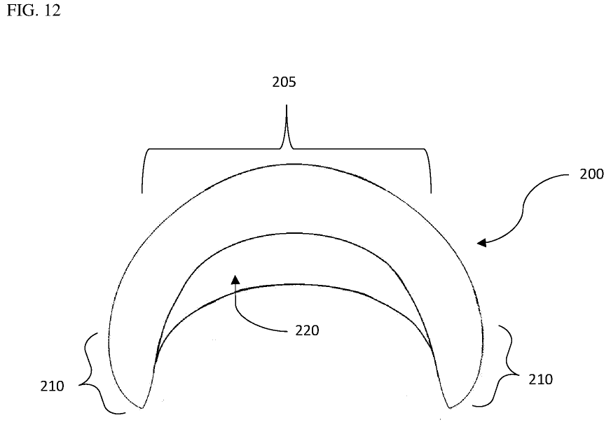

In certain embodiments, the chamber element is in the approximate shape of a spherocylinder bisected along its longitudal axis FIG. 10, 215 to form an approximate half or partial spherocylinder 200. As used herein a spherocylinder is a three-dimensional geometric shape defined by a half cylindrical center segment FIG. 9, 205 capped on each distal end with approximate quarter-spheres FIG. 9, 210. As used herein, the longitudal axis is defined as the long axis of the spherocylinder beginning and finishing at the distal ends of the quarter-spheres of the spherocylinder. As used herein, a half cylinder is defined as three-dimensional geometric figure with parallel sides and a half circular or half oval cross section. An approximate quarter-sphere comprises a three-dimensional geometric figure in the form of an approximate sphere bisected approximately in quarters. The flat surfaces of the approximate quarter spheres are adapted to conform to the ends of the half cylindrical center segment and when the spherocylinder segment is bisected along the longitudal axis, FIG. 11, 215, the edge of the chamber element is designed to conform to a target therapy area forming a sealed cavity between the surface of the user and the device. FIG. 12, shows a partial spherocylinder 200, bisected along the longitudal axis 215 with a central cylindrical segment 205 and quartered spherical ends 210

In additional embodiments the chamber element is approximately spherocylindrical in shape. As used herein, an approximately spherocylindrical shape can define a chamber that is not exactly planar, but concave or convex. FIG. 12. Depicts a spherocylinder 200, as bisected in FIG. 10, 215, providing for a curvature approximately perpendicular to the longitudal axis of the chamber.

In preferred embodiments, the chamber element is flexible and self-supporting. As used herein, flexible refers to the chamber's ability to move, twist and stretch allowing for a more comfortable fit and interface between the device and user. As used herein, self-supporting refers to a property of the chamber to substantially maintain shape over a range of negative pressures and have a resilient memory such that the chamber element is biased towards returning to it original shape following an event that causes chamber collapse or other shape change. As used herein substantially maintain shape is defined as 0% to 10% deflection of the chamber element. As used herein, deflection of the chamber element and or deflection of the arch is defined as the movement of the arch and or peripheral edges of the cylindrical portion of the chamber element. Inward deflection may cause a decrease in the chordal span 140 and or collapse of the chamber element inward and outward deflection may cause an increase in the chordal span 140 and or collapse of the chamber element outward.

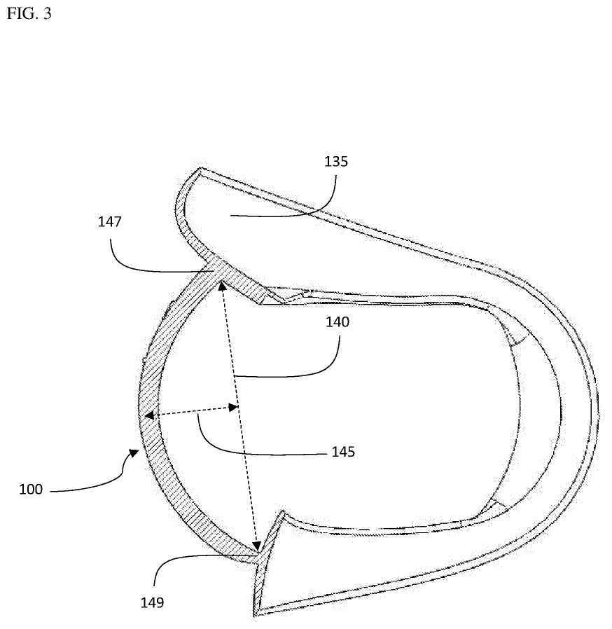

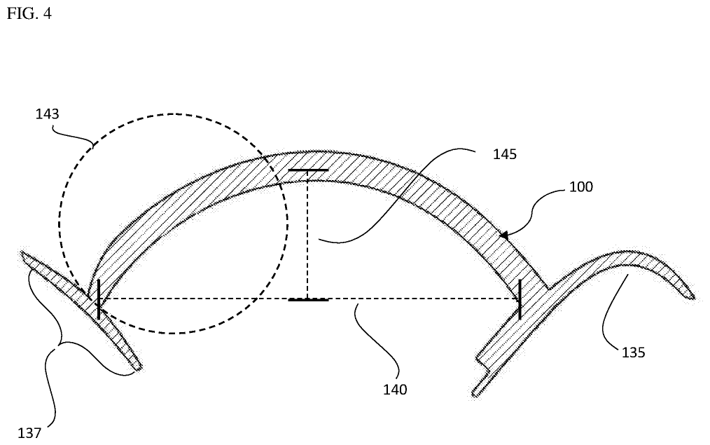

The partial cylindrical center segment of the chamber element in the form of an arch, may contain a height FIG. 3, FIG. 4, 145 and chordal span FIG. 3, FIG. 4, 140 that provide for self-supporting properties. As used herein an arch is defined as the curved shape of the approximate cross section of the partial cylinder of the chamber element, supporting the force of the negative pressure over a range of negative pressures. As used herein, negative pressure is defined as a negative pressure within the chamber element as compared to atmospheric pressure outside the chamber element. The height of the arch is measured from the approximate apex of the arch to the line of the chordal span, FIG. 3/FIG. 4, 145. The chordal span FIG. 3/FIG.4, 145 of the arch is measured from one spring point of the arch to the opposing spring point wherein the spring point is defined as the base of the arch at a point along the peripheral edge of the chamber element. The chordal span line as used herein is a line spanning from one spring point toward the other and perpendicular to the line of the height. For example, FIG. 3/FIG. 4, 147, 149 shows the upper and lower spring points respectively wherein the upper spring point is closest to the chin cup and the lower spring point is closest to the laryngeal prominence and the chordal span/chordal span line 140.

In certain embodiments, the height of the arch and the ½ span of the arch are equivalent creating a symmetric half-tube however in preferred embodiments the ratio of the height of the arch to the ½ span of the arch is ranges from approximately 0.65 to 0.85 and more preferably approximately equivalent to 0.75.

The chamber element may further comprise a material composition selected to be flexible but have a minimal bending stiffness. As used herein, minimal bending stiffness is defined as the resistance of the chamber to bending, deformation and or collapse when at least a therapeutic level of negative pressure is applied. The flexible material may be of any suitable composition, for example silicone rubber, pvc, polystyrene, etc wherein the thickness of the chamber can be varied in accordance with the bending stiffness's of the material used.

The chamber element is designed to approximately conform to a therapy area on a user. The term: “approximately conform to” an anatomical location refers to contacting closely to the actual location, shape or size but perhaps not necessarily completely, accurately or exactly. In certain embodiments the chamber element is configured to follow the contour of the therapy device which is designed to approximately conform to an individual from approximately a first location corresponding to a first gonion on one side of the individuals mandibular body to a second location corresponding to the individuals mental protuberance to a third location corresponding to the second gonion on the opposite side of the individual's mandibular body and a fourth location corresponding to the individuals thyroid cartilage further configured return to approximately the first location corresponding to the first gonion.

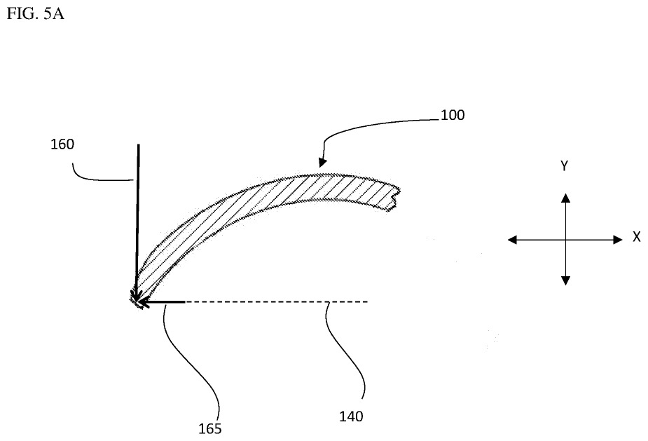

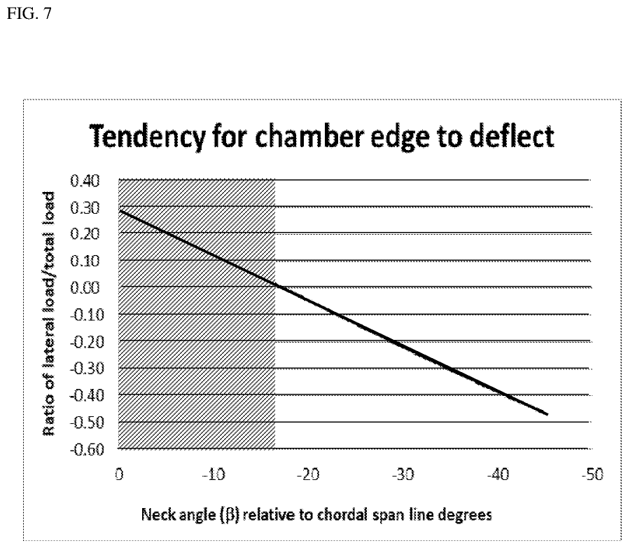

The gonion, as used herein, describes the approximate location on each side of the lower jaw on an individual at the mandibular angle. The mandibular protuberance, as used herein, describes the approximate location of the chin, the center of which may be depressed but raised on either side forming the mental tubercles. The thyroid cartilage, as used herein, describes the approximate location of the large cartilage of the larynx in humans. A region approximately corresponding to the thyroid cartilage is depicted by the dotted lines in FIG. 8, 180; a region approximately corresponding to the gonion is depicted by the dotted lines in FIG. 5. 185; and a region approximately corresponding to the mental protuberance is depicted by the dotted lines in FIG. 8, 190. Note that FIG. 8 show a right profile, and a similar region is present on the left profile.

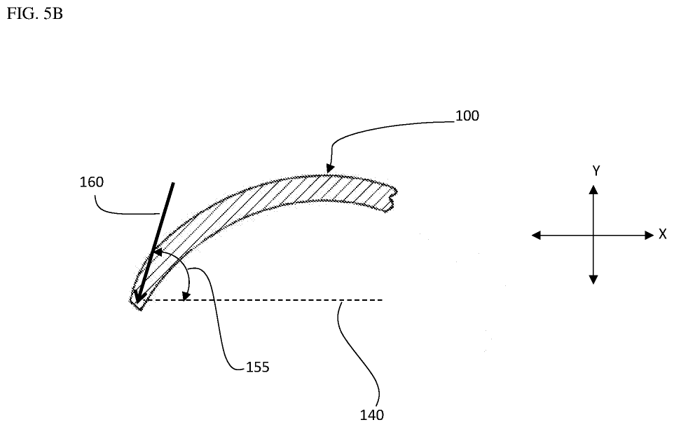

The chamber element makes contacts the skin of the user along its peripheral edge, enclosing an approximate target therapy area. The varying surface features of the user provides for a range of angles observed between the chamber element and the point of contact on the user. This measurement, made from the inside of the chamber element to the skin of the user is defined herein as the neck angle, FIG. 6, 175. In preferred embodiments, the chamber element is designed to enclose the approximate target therapy area and provide for a neck angle measurement between −18 and −24 degrees.

Application of negative pressure within the chamber element when the therapy device is applied to a sealing surface, for example the approximate target therapy site of the user, provides a range of measurable force vectors perceived by the user as a contact pressure between the peripheral edge of the chamber element and the skin of the user and when the chamber element is attached to a flange element, along the interface of the flange element and the skin of the user. Load/force vectors as used herein are the observed forces and the directional bias of the forces. Bias of the load vectors is dictated by the shape of the chamber element, angle at which the chamber element or a chamber element attached to a flange element contacts the surface of the user and neck angle at the point of contact of the chamber element to the skin of the user. Examples of force vectors can be seen in FIG. 5B, wherein 150 indicates the approximate total chamber load vector. As used herein the approximate total chamber load vector is defined as the total magnitude of force and direction in which the force is applied.

The total magnitude of force can be calculated from the vertical and horizontal force vectors. FIG. 5B shoes a partial side view of an approximate half of the device as called out in FIG. 4, 143 with the flange element removed. The vertical force vector 160 (on the Y-axis FIG. 5A) is defined as the force vector parallel to the line of the chamber height measurement line 145 and the horizontal force vector 165 (on the X-axis FIG. 5A) is defined as the force vector parallel to the chordal span line 140. In the instant invention, the shape of the chamber is such that the magnitude of the vertical force vector 160 is greater than that of the horizontal force vector 165 (As see in FIG. 5A 160, 165) when a therapeutic level of negative pressure is applied. The larger vertical force vector 160 provides for a total load vector that is mostly biased in the vertical direction. FIG. 5B, shows the total load vector 150 as calculated as the sum of the vertical and horizontal force vectors providing for a total force vector angle (α) 155 as measured from the chordal span line 140. In embodiments of the invention the total load vector angle (α) 155, as measured from the chordal span line 140 is between approximately 65 and approximately 90 degrees and between approximately 65 and approximately 79 degrees and between approximately 70 and approximately 74 degrees and preferably approximately 72.5 degrees.

Bias of the load vectors can cause deflection of the chamber in a range of negative pressures. Deflection as used herein is the movement of the chamber element inward or outward upon the application of negative pressure. In certain embodiments, deflection of the chamber can be minimized via materials with very stiff properties, however it is an object of this invention to provide for a flexible chamber with minimal bending stiffness for better comfort and compliance. These chambers may also be thinner allowing for material savings, weight reduction and more flexible devices. Further, a taller chamber/arch, may provide for a larger volume for tissue to ingress when a negative pressure is applied, but this taller shape may bias the load vectors inward prompting collapse of the chamber via the arch's chordal span dimension. A shallower chamber/arch, may bias the load vectors outward, tending to increase the chordal span dimension at the cost of possibly not allowing enough tissue movement into the chamber.

The tendency of the chamber arch to deflect can be calculated as a ratio of the total load vector FIG. 5C, 150 to the lateral load vector FIG. 6, 165, wherein the total load vector (FBx) is defined by the equation:

FBx=P((S²)+(H²))/2H
wherein P is the negative pressure within the chamber, S is the length of ½ the chordal span (FIG. 4, 140) of the chamber arch and H is the height of the chamber arch (FIG. 4, 145) measured from the perpendicular line of the chordal span line (FIG. 4. 140),
wherein the lateral load vector (Fal) is the force vector observed on the plane parallel to the contact surface of the skin FIG. 6, 170 on the P-axis. The lateral load vector(Fal) is defined by the equation:

Fal=FBx*COS(α−β)
wherein α is the resultant total force vector angle (FIG. 5, 150, 155) calculated by the arctangent of the vertical load vector (FIG.5A, 160) divided by the horizontal load vector (FIG.5A, 165),
wherein β is the angle of the neck (FIG. 6, 175) as measured between the line parallel to the contact surface of the skin 170 to the chordal span line 140.
wherein the vertical load vector (FAy), FIG. 5A 160 is the negative pressure multiplied by the ½ the chordal span (FIG. 4, 140)
wherein the horizontal load vector (FAx), is the total load vector (FBx), minus the sum of the negative pressure within the chamber (P), multiplied by the chamber arch height (H),

FAx=FBx−(P*H).

In certain embodiments, the chamber element 100 or a flange element 137 attached to the chamber element along the circumferential edge of the chamber element makes contact with the individual at an approximate location corresponding to the upper airway of the individual. The point of contact of the chamber element to the skin of the individual may comprise one or a range of neck angles (b), FIG. 6, 175. As used herein the neck angle (β) FIG. 6, 175 is the angle as measure from the chordal span 140 to a line parallel to and corresponding to contact point or surface of the chamber element to the skin or the flange attached to the circumferential edge of the chamber element and the skin. In certain embodiments, the neck angle or range of neck angles is approximately between −18 and −24 degrees is present. As used herein, the neck angle is defined as the angle measured between the line of the chordal span FIG. 6, 140 and a line parallel to the contact surface of the individual 170.

Application of at least a therapeutic level of negative pressure within the chamber element causes a measurable value of a tendency to move of the chamber element and may cause deflection of the arch. Tendency to deflect and deflection of the arch is caused by the translation of the load vector through the arch of the chamber element on the user. As used herein, tendency to deflect is defined as the ratio of the total load vector to the translated outward load vector. As used herein deflection is the movement of the arch of the chamber element inward or outward at or nearest to the peripheral edge of the chamber. The tendency to move does not always indicate chamber movement as, by way of example, both a rigid chamber and flexible chamber both have a tendency to deflect however little to no deflection of the rigid chamber would be expected due to the material properties of the rigid chamber. It is an object of this invention to provide for a flexible chamber element that is self-supporting and resistant to deflection and/or collapse. In preferred embodiments, the tendency to move ranges from approximately 0 to approximately −0.3 and the inward deflection of the arch is between approximately 0 and approximately 0.4 inches.

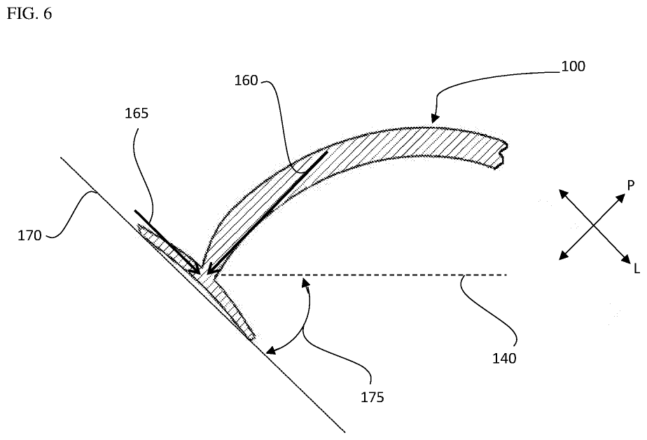

By way of example, as illustrated in FIG. 7, that shows a range of neck angles on the X-axis and the ratio of the total load vector to the translated load vector on the Y-axis, a flexible chamber element with a flexible central region of the partial cylinder, wherein an arch section with a height of approximately 1.02 inches, a ½ chordal span length of approximately 1.39 inches, a thickness of approximately 0.17 inches, a material composition of Shore A durometer 40 silicon rubber providing an approximate chamber stiffness of 0.146 N/mm, under an approximate negative pressure of 0.43 psi, is applied to external surface of an individual, wherein at the point of contact of the chamber to the skin of the individual, a neck angle of approximately between −18 and −24 degrees is present, provides a ratio of the total load vector to the translated outward load vector ranges that ranges from approximately 0 to approximately −0.3 and the inward deflection of the arch is between approximately 0 and approximately 0.4 inches.

The flange element preferably comprises a flexible, elastic material that can be uniform in thickness and width but also vary in thickness and width to achieve the structural properties desired at locations along the contact surface of the therapy device. The flange element may further contain a curved profile on the top surface and a flat profile on the bottom surface. The top surface of the flange element being that which makes contact with the chamber element and the bottom surface of the flange element being that which makes contact with the skin of the user. As used herein a “curved profile” describes the shape of a flange element that is thicker at the junction between the flange element and chamber element and thinner towards the outer edges thereof. The flange element may contain edges that taper outwardly for avoiding skin deformation and cutting associated with hard sharp edges.

Optionally, an adhesive layer is located on the surface of the flange element that makes contact with the user. These elements are configured to maintain an approximate uniform contact pressure with minimized pressure variations along the skin of an individual through all points of contact of the therapy device on a patient. By “minimized pressure variation” means a pressure at any point between the contact surface of the flange element and the patient's tissue varies by no more than about 20%, and preferably no more than about 10% or about 5%, from the average pressure across the entire contact surface. The outer contact surface, as used herein, is the surface of the flange element of the therapy device that makes contact with the skin of the individual forming the contact and sealing surface of the therapy device.

In certain embodiments, the flange element of the invention provides a contact interface of a negative pressure therapy device configured to conform to a continuous contact area on the individual at the external area of the neck approximately corresponding to the anterior triangle of the neck. The term “approximately corresponding to” an anatomical location refers to contacting closely to the actual location, shape or size but perhaps not necessarily completely, accurately or exactly.

Most preferably, the flange element is configured to follow the contour of the therapy device which is designed to approximately conform to an individual from approximately a first location corresponding to a first gonion on one side of the individuals mandibular body to a second location corresponding to the individuals mental protuberance to a third location corresponding to the second gonion on the opposite side of the individual's mandibular body and a fourth location corresponding to the individuals thyroid cartilage further configured return to approximately the first location corresponding to the first gonion.

In certain embodiments, the negative pressure therapy device of the present invention is a chamber element, approximately an elongated dome, oval appearance, with a curvature from the middle of the chamber that creates a collar to cover an area over the upper airway of an individual. In preferred embodiments, the negative pressure therapy device contains structural elements adapted to guide correct placement and orientation of the device on the user, for example a chin cup element. As used herein a “chin cup” refers to a discreet feature on the negative pressure therapy device which provides a recess configured to receive the chin of the wearer when the negative pressure therapy device is properly mated to the wearer. During application of the negative pressure therapy device, the chin cup provides a consistent point of reference on which the negative pressure therapy device can mate with the wearer. The shape of the chin cup may vary to allow for anatomical variation in patients. For example, the chin cup may be somewhat deeper for use in a subject having mandibular prognathia; somewhat shallower for use in a subject having mandibular retrognathia; or somewhat larger in volume for a subject having macrogenia.

In various embodiments, the present invention comprises a symmetric negative pressure chamber element with a flat contact surface adapted to fit to a flat uniform surface and to provide minimized pressure variation throughout all points of contact when a negative pressure is applied. In other various embodiments, the present invention comprises a negative pressure chamber element with a contact surface configured to adapt to the inherent anatomical variations of an individual's face. The curved, “wraparound” shape that the negative pressure therapy device must assume can cause the “station load” through different contact points to vary in the absence of the design features described herein. For example, absent a feature or features designed to accommodate for station load variation, at points furthest from the center of the chamber of the therapy device, toward the narrow end portions of the oval, the station load decreases due to a lesser negative pressure cross section over the contact point(s). As used herein, “station load” is the force or pressure which is applied at a discreet area of contact of the device (a “station”) on the skin of an individual when the device is mated to the individual and a therapeutic level of negative pressure is applied.

In certain embodiments, the present invention comprises a chamber element having a shape that when unloaded, i.e. not on the patient, spacing between the first and third locations is narrower than the spacing that is obtained when the chamber element is mated to the individual and a therapeutic level of negative pressure is applied. The narrower spacing of the unloaded device creates a preload force that is applied to the individual by the chamber element prior to the application of negative pressure.

As discussed herein, the flange element of the instant invention forms the interface between the chamber element of the therapy device and the contact surface of the individual. The chamber element of the instant invention forms the dome/chamber element of the therapy device. These elements comprise structural features that provide minimized pressure variation at stations where contact pressure variation can occur as a result of either anatomical variation, tissue variation, inherent therapy device design, and or movement during usage. The flange element and chamber element thereby providing features to the therapy device to minimize peak contact pressure values, minimize the variance from station to station and equalize the contact pressure of the therapy device when a therapeutic level of negative pressure is applied to provide an effective seal.

The term “seal” as used in this context is not to necessarily imply that a perfect seal is formed between the therapy device and the contact surface of the individual. Rather, a “seal” is a portion of the device which mates to the wearer and maintains a therapeutic level of negative pressure. A certain amount of leakage at the seal may be tolerated so long as the desired negative pressure can be achieved and maintained. Preferred operational negative pressure levels are in a range of between 7.6 cm to about 61 cm of water. Preferred forces applied to the user's neck tissues in order to assist in opening the upper airway passages are in a range of about 0.5 kilogram to about 6.68 kilograms. The term “about” and “approximately” as used herein with regard to any value refers to +/− 10% of that value.

The elongated dome/chamber element provides a finite volume which must be evacuated to deliver the desired partial negative pressure level. Once generated, the partial negative pressure will decay at a rate which is primarily controlled by leakage of air into the chamber element past the seal and or features integrated into the chamber element to provide airflow. In certain embodiments, the chamber element encloses a volume of between about 8.2 ml and 196.6 ml. Preferably, the leakage is no more than between about 0.08 ml/min and 8.2 ml/min, and most preferably between about 0.16 ml/min and 1.6 ml/min.

The therapy device may comprise one or more vent elements. As used herein a vent element is an aperture through the therapy device that provides airflow in to the chamber element when the chamber element is mated to the individual and a therapeutic level of negative pressure is applied within the chamber element. The aperture(s) can be in any suitable location on the device however in some embodiments the aperture(s) may be located at the top of the chamber element closer to locations one and three on the individual. The vent element(s) may simply be an aperture such that when the chamber element is mated to the individual and a therapeutic level of negative pressure is applied an airflow between about 10 mL/min and about 60 mL/min is achieved or an aperture through which a filter element can be inserted to create filtered airflow such that when the chamber element is mated to the individual and a therapeutic level of negative pressure is applied an airflow between about 10 mL/min and about 60 mL/min is achieved. The filter element can be a replaceable element and comprise a pore size of between about 0.25 μm and 0.1 μm or less such that when the chamber element is mated to the individual and a therapeutic level of negative pressure is applied an airflow between about 10 mL/min and about 60 mL/min is achieved.

The present invention provides both sufficient regional, and overall, compliance of the therapy device such that local bottoming/regional collapse of the device does not occur under load. As used herein, “regional compliance” of the device refers to the ability of individual stations of the device to accommodate a therapeutic level of negative pressure without complete compression at that station. As used herein, “overall compliance” of the device refers to the ability of the device to accommodate a therapeutic level of negative pressure without complete compression of the device. Further, bottoming or “regional collapse”, as used herein, is defined as a complete or near complete compression of the device that its resistance to further compression is no longer possible. This results in a hardening of supporting structure(s) by the flexible portions of the device under a heavy load, and loss of comfort by the wearer.

The flange element and chamber element are designed to create uniform contact pressure onto the skin of the user when a therapeutic level of negative pressure is applied. The flange element is preferably a perpendicular width (wide and narrow) and thickness to achieve the desired contact pressure properties. The perpendicular width component is the total width of the flange element, from the tip of the outside edge of the flange element through the root and to the tip of the inside edge of the flange element. The width of flange element may vary along the peripheral axis of the contact area of the flange element to accommodate for station load variations due to non-uniform shape of the therapy device that contains a chamber element, that is oval in shape and further contains a central bend to accommodate the mating surface on the neck of the patient corresponding to approximately the upper airway and maintain a constant contact pressure of the negative pressure therapy device.

In various embodiments, locations on the flange element of the device may be substantially wider than other locations. In one aspect, the total flange element width may vary from approximately 28.0 millimeters to approximately 17.0 millimeters. “Substantially wider” as used herein refers to an increase in width of at least about 10%, more preferably at least about 20%, and still more preferably at least about 30% or more from one location to another, for example in an embodiment of the invention the width of the flange element at the fourth location corresponding to approximately the middle of the neck of the user is approximately 39% wider than the first and third locations that corresponding to the mandible and gonion regions of the user. Wider sections may be found in regions where a larger load displacement is needed for example at the second and fourth locations and narrower sections may be found in regions where smaller load displacement is needed for example at the first and third locations on the user.

The thickness of the flange element may also vary along the perpendicular width along the circumference of contact surface of the therapy device to accommodate for anatomical variation and varying negative pressure cross section. As used herein, thick or thin, describes the distance between the surface of the flange element contacting the individual and the (distal) surface of the flange element contacting the chamber element of the negative pressure chamber element of a negative pressure therapy device. The thickness of the flange element at the root may vary from approximately 4.5 millimeters to 1.0 millimeters at the inside of the root and 3.0 millimeters to 1.2 millimeters at the outside of the root. For example, the thickness of the flange element at the junction at the first and third locations on the user may be about 1.6 millimeters inside the root and 2.10 millimeters outside the root. These exemplary measurements depend to some extent on the durometer of the material chosen, in this case a Shore A durometer of between about 30 and about 50. The skilled artisan will understand to adapt these measurements to different materials making up the flange element and chamber element.

In certain aspects, locations on the flange element of the device may vary in thickness such that some portions are substantially thicker than others. For example, locations of the flange element may vary in thickness such that on location is substantially thicker than another. As used herein, “substantially thicker” refers to an increase in thickness of at least about 20%, more preferably at least about 30%, and still more preferably at least about 50% or more. For example, in an embodiment of the invention the thickness at approximately the second location is approximately 64% thicker that the first and third locations and the first and third locations are approximately 30% thicker than the fourth location.

The thickness of the flange element may further taper outwardly from the root location to a final flange element thickness of approximately 0.7 millimeters to approximately 0.1 millimeters. The taper may begin at the root continuing to the inside or outside edge of the flange element or the taper may also begin at points about 10%, 15%, 20%, 25%, 30%, 35%, 40%, 45%, 50%, 55%, 60%, 65%, 70%, 75%, 80%, 85%, 90% or 95% away from the tip of the flange element and continue to the inside or outside edge of the flange element to a desired final thickness of approximately 0.7-0.1 millimeters. The taper of the flange element at its inner and outer edges assisting in the elimination of edge effects allowing for minimized tissue irritation and damage. As used herein, “edge effects” refer to the irritation, (redness, swelling) of tissue caused by prolonged contact pressure of a sharp edge on the skin. The tapering of edges provides for a more flexible and softer edge of the flange element.

The chamber element is stiff along its length and the flange element will not appreciably deflect longitudinally. Therefore, in addressing the dynamic shape of the target therapy area, regions of the therapy device contain accommodating design features, for example, the variations in the width and thickness of the flange element, that are designed to minimize high pressure points and eliminate contact pressure variations of the therapy device along its contact surface when placed on the user and a therapeutic level of negative pressure is applied.

In regions where the flange element contacts a substantially flat surface of the user, the chamber element and flange element can act as an “I-beam” where the force exhibited by the flange element on the user is a more linear downward force and cantilever-like. The flange element inside and outside the root point of the chamber element flex according to the thickness of material with the tapered ends of the flange element flexing the most creating a soft transition on the skin of the user eliminating edge effects as above. As used herein cantilever-like forces are a measurement of the downward force of the chamber element divided by the area of the flange element at a given point. By way of example, in regions where the flange element lays flat across the skin, cantilever forces can be balanced by altering the width and thickness of the flange element, for example where there is a high negative pressure cross section and where larger load distribution is desired (ie. lower contact pressure), a flange element with a larger perpendicular width may be utilized and similarly in regions where a smaller load distribution is desired (ie higher contact pressure) a flange element with a smaller perpendicular width may be utilized

The thickness dimensions of the flange element can give the flange element properties such that in portions of the device, if the flange element is too thin, though it may be very flexible it will have little to no load distributing properties, can bottom out creating point(s) of high contact pressure from the root of the chamber element resulting in leaks and/or discomfort. If the flange element is too thick it will affect its ability to change direction for example be unable to conform to the acute change from the surface of the neck over the mandible toward the ear for example and further allow for an undesirable level of sheer or lateral movement. Wherein sheer or lateral movement is defined as movement along the plane parallel to the skin of the user where the chamber element or a chamber element comprising a flange element make contact. In a similar fashion, if the width of the flange element is too small it can create a point(s) of high contact pressure and too wide it may create unnecessary bulk affecting fit and effective therapy area. Transition in widths taper gradually and the aspect ratio minimizes positional instability and optimizes flexibility.

In regions where the flange element contacts a curved surface of the user, for example around the chin and over the mandible, the forces observed contain an additional hoop-like force component as the flange element bends around those features. “Hoop-like forces” as used herein describe the distribution of force exerted circumferentially, for example, as the flange element travels around location four of the of the user the curvature adds additional stiffness to the flange element inside and outside the root of the chamber element. In these regions where the added force component of hoop loads exists, the thickness of the flange element may be decreased and the perpendicular width of the flange element may be increased to effectively distribute the load of the chamber element and minimize contact pressure variation from station to station when a therapeutic level of negative pressure is applied.

The term “contact pressure” as used herein refers to a pressure imparted on the surface of the skin by the contact surface of the device. Its value can depend on the negative present as well as the structural characteristics of the flange element such as the perpendicular width and surface area of the contact surface, and can vary at different locations on the flange element.

A larger “perpendicular width” of a contact surface (meaning the direction that is perpendicular to the longest axis of the contact surface, which longest axis may be curved) will have a lower overall contact pressure under the same negative pressure as a contact surface with a smaller perpendicular width due to the increased surface area at that particular station of the contact surface. Therefore, in regions where the chamber station pressure load is low, the contact surface of the flange element can be designed to be of a smaller perpendicular width to effectively increase and “balance” the contact pressure and in regions where the chamber station pressure is high, the contact surface of the flange element can be designed to be of a larger perpendicular width to effectively decrease and balance the contact pressure where the chamber station load is high.

In certain embodiments, the location of the chamber element on the flange element (the root location) may vary from the mid-point, inward or outward to further aid in equalizing the contact pressure of the therapy device on the user when a therapeutic level of negative pressure is applied creating and maintaining the balance point of the flange element on the user. For example, movement of the root of the edge of chamber element on the flange element outward from the mid-point of the flange element effectively increases the negative pressure cross section and therefore effective contact pressure of the therapy device at that point when a therapeutic level of negative pressure is applied. Movement of the edge of the chamber element inward has an opposing effect, providing a larger portion of the flange element exposed outside the root location and therapy area decreasing the negative pressure cross section. In regions where higher contact pressure is needed, for example where the device approaches the ear of the user, the chamber element location can be biased on the flange element toward the outer edge increasing the negative pressure cross section and effective contact pressure at that point.

In certain embodiments, the chamber element may contain features that further aid in the prevent regional collapse, bottoming and transfer of force from one region to another of the therapy device. Absent local points of flexibility, a rigid chamber element may experience situations where external pressure could cause a point of high contact pressure for example upon application of a force, by rolling on to a pillow etc., on the device causing a bottoming event or further a rigid chamber element may experience situations where external pressure on the device on one side causes a transfer of force to the opposite side of the device. Events such as these may cause discomfort, dislodging of the device or both.

In one aspect, the chamber element is formed with one or more recesses positioned therein. In preferred embodiments, the chamber element may contain several recesses. As used herein, a recess refers to a space created by molding a portion of the chamber element thinner than the surrounding material such that when a force is applied the thinner material is able to bend, flex or compress and rebound. The recess is preferably of a thickness to provide the desired properties without rupturing or causing collapse of the chamber element when a therapeutic level of negative pressure is applied.

In certain embodiments, one or more recesses are located at points in the chamber element of the therapy device where additional flexibility of the device is needed to reduce point loads where the flange element contacts the user, for example at regions where the device needs to follow anatomical features that rapidly change direction or are particularly hard. By way of example the chin feature, mental tubercles and or the lower neck feature at or around the laryngeal prominence, nearest the second and fourth locations on the user respectively, represent such features. The recess can be of any appropriate shape however in some embodiments the compressible recess is approximately crescent in shape. As used herein a “crescent shape” is described as generally the shape produced when a circular disc has a segment of approximately another circular disc removed from its edge so that what remains is a shape enclosed by two circular arcs of different diameters which intersect at two points. This feature provides a region of compressibility that tapers from regions where larger compression is needed (the middle of the crescent) to regions where less compression is needed (the outer points of the crescent)

In certain embodiments, the therapy device may contain one or more first recesses located at approximately at a junction formed between the flange element and the chamber element at approximately the flanks of the chin feature of the chamber element near the mental tubercles of the user, closer to a point nearer to the second location of the user than any of the other locations providing for a first hinge region. Located approximately at a junction, as used herein describes a location closer to one point verses another nearer to where but perhaps not exactly, for example, “one or more first recesses located approximately at a junction formed between the flange element and the chamber element corresponding to the second location” indicates the location of the recess being near a point where the chamber element meets the flange element however not exactly at said junction. As used herein the “flank of the chin” describe the points of the chin where it bends from the front of the face of the user and progresses backwards along the mandible toward the gonion. The bend, creating an anatomical feature where a compressible recess may be beneficial. As used herein the first hinge region is defined as point on the therapy device that can pivot creating a decoupling of one side of the device from the other side of the device.

In certain aspects a recess may be of approximately 0.75 inches in length from tip to tip of the crescent and approximately 0.125 inches in width at the center of the crescent. Further as the compressible recesses flank the chin feature of the device, the compressible recesses may begin at a location approximately 0.5635 inches from the vertical center of the device and progress following approximately the shape and contour of the edge of the chamber element and contact surface of the flange element.

In certain embodiments, the therapy device may contain one or more second compressible recesses within the chamber element approximately positioned at a junction formed between the flange element and the chamber element at approximately the lower neck portion of the chamber element nearest the fourth location of the user providing for a second hinge region within the chamber element. The compressible recess may be of a crescent shape that approximately follows the edge of the chamber element and radius/contour of the contact surface of the flange element. The compressible recess may be of approximately 1 inch in length from tip to tip of the crescent and approximately 0.25 inches in width at the center of the crescent.

In certain embodiments, the first and second compressible recesses provide for first and second hinge regions that are configured to reduce the transmission of deformational strain within the chamber element relative to a chamber element lacking the first and second hinge regions. A hinge region, as used herein describes a region of the device where one side can bend or pivot independent of the opposite side. The term “deformational strain” as used herein refers to a force applied on the therapy device that causes collapse of the chamber element or disengagement of the device from the individual during use. By way of example, if a user rolls onto a pillow on one side of the device, deformational strain may be transmitted to the other side, causing the device to lift off of the face. The hinge region(s) alone or in combination with other design features described herein effectively allows for a decoupling of force from one side of the device and maintains the device's position on the user and therapy.

The term “balance” as used herein refers to the contact pressure of the therapy device being approximately equal at each station of the contact surface. This contact pressure is proportional to therapy negative pressure levels relative to the contact area of the therapy device. For example, in a comparison, a larger contact area vis. a smaller contact area, under the same therapy negative pressure level will provide for lower contact pressure of the therapy device respectively. In an embodiment of the invention the contact area of the flange element relative to the therapy area provides for a contact pressure that may range from approximately 0.9 to approximately 1.5 times the negative pressure level and in a preferred embodiment the contact pressure of the flange element is approximately 1.2 times greater than therapy negative pressure levels.

The chamber element is operably connected to an air pump to produce the therapeutic level of negative pressure within the chamber element. The air pump can be of any type to suitable to produce the therapeutic level of negative pressure for example positive displacement pumps, impulse pumps, velocity pumps, etc which can include manual squeeze bulbs, rotary pumps, lobe pumps, oscillatory pumps etc. In certain embodiments, the air pump comprises a piezoelectric material configured to provide an oscillatory pumping action wherein the oscillatory pumping motion operates at a frequency greater that 500 Hz.

The air pump may be a separate component connected to the chamber element via a hose or tube, or may be configured integrally to the chamber element. The air pump can be connected to the chamber element in any suitable fashion for example an air pump may external the chamber element and connected via a hose or tube, stationary, for example bed-side, or battery powered and wearable by the patient. In certain wearable aspects, the air pump is configured to be integral to the chamber element. For example, the air pump may be configured to insert into a sealable aperture on the chamber element, the air pump tightly fitting through the aperture creating a seal. As used herein a sealable aperture is an opening through an element of the apparatus that can be closed or sealed from one side or the other with another element of the apparatus creating an air-tight or water tight seal.

In a preferred embodiment, a seal is created via surfaces designed to receive an O-ring. As used herein an O-ring is a gasket in the form of a compliant sealing ring made of a pliable material designed to be compressed during assembly creating a seal at the interface. In certain aspects, a complaint sealing ring feature may be an integral, unitary or discrete part of the air pump, the chamber element or both. In certain embodiments, the compliant sealing ring is provided as a component of the air pump. In a preferred embodiment, the compliant sealing ring feature is a molded feature on the inner circumference of the aperture of the chamber element. In a preferred embodiment, the compliant sealing ring feature comprises a lip seal.

In certain aspects of the invention, one or more tangs, tabs and or recesses are present on the chamber element, flange element and or air pump element of the therapy device, which provide one or more guidance feature(s) to ensure a proper orientation of, or mating between one or more device elements. The tangs, tabs and or recesses can be utilized as part of a sensor system to determine various parameters related to use of the therapy device. These parameters can include, but are not limited to, compatibility of the particular air pump element with the therapy device element (e.g., acting as a recognition feature) and correct placement of the air pump element into the aperture of the chamber element. For example, one or more of these tangs, tabs or recesses can be located on the chamber element as a guidance feature for the air pump element such that a recess on the air pump element or chamber element accepts the tang or tab element on the chamber element or air pump element only when the air pump element is inserted through the sealable aperture in the correct orientation. This list is not meant to be limiting.

In certain embodiments, together or with one or more of the foregoing, a material, which will act as an adhesive layer between the flange element of the therapy device and the user, is applied to the outer contact surface of the flange element. The purpose of the adhesive layer is to provide a sealing, cushioning and or sheer absorbing element to the flange element. As used herein sheer refers to sheer strain which is a deformation of a material in which parallel surfaces can slide past one another, for example the contact surface of the flange element and the skin of the user.

The adhesive layer further must preferentially adhere to the outer contact surface of the negative pressure therapy device and provide a sufficient level of “tack” such that a releasable mechanical anchoring of the therapy device to the skin of the user is achieved. Tack, as used herein, refers to a material property at the interface created between the adhesive layer and the device, and the skin of the user at the other interface created between the user and the device.

The adhesive layer may be applied to the contact surface area of the negative pressure therapy device in any suitable method including but not limited to spraying, painting, placing, etc., in single or multiple layers to achieve the desired cushioning and sealing properties including but not limited to thickness, hardness and tack for example. In additional embodiments the adhesive layer may be single layer of a uniform thickness or a single layer of a non-uniform thickness covering the contact surface of the negative pressure therapy device. In further embodiments, the adhesive layer may contain a series of parallel adhesive beads spanning the circumference of the contact surface of the negative pressure therapy device wherein the beads can be of a uniform or non-uniform thickness and of a like or varying adhesive and or gel-like material to achieve the desired cushioning and sealing properties.

In certain embodiments, the adhesive layer is present on the contact surface of the negative pressure therapy device at a thickness falling within a range of approximately 0.005-0.060 inches. In certain embodiments, the adhesive layer is present on the contact surface of the negative pressure therapy device at a thickness falling within a range of approximately 0.010-0.050 inches. In further embodiments, the adhesive layer is present on the contact surface of the negative pressure therapy device at a thickness falling within a range of approximately 0.020-0.040 inches.

The adhesive layer may be achieved by using various materials, such as, but not limited to gel, elastomer, viscous solutions, foams and materials of the like. These materials can be of any chemical composition which provides the necessary end use properties (i.e. tack, firmness, medical clearance, etc.). These materials include, but are not limited to polyurethanes, silicones, acroylnitrile butadiene styrene (ABS), hydrogels, and the like. In preferred embodiments, the adhesive layer should have a hardness as measured by ASTM-D2240-00 (American Society for Testing Materials) of between 0 and 50, more preferable between 5 and 30 most preferable between 5 and 15. In certain embodiments the adhesive layer is made of a silicone gel material. The silicone can be any organosilicone which yields the desired properties although polydimethylsiloxane (PDMS) is often chosen.

The adhesive layer may be applied directly to the outer contact surface of the flange element to a desired thickness or in combination with one or more primer layer and or one or more primer layers in combination with one or more adhesion or binding promotor layers to create a lamination stack of materials to a desired thickness. As used herein a “primer” is a substance used as a preparatory coating, acting as a joining surface between the contact surface of the negative pressure therapy device and adhesive layer or an adhesion promoting layer and the adhesive layer. Further, an adhesion promoting layer is a substance used as a coating to preferentially adhere the adhesive layer to the contact surface of the negative pressure therapy device and or the primer layer that is applied to the outer surface of the negative pressure therapy device.

By way of example, a primer layer may be applied to the contact surface of the negative pressure therapy device to a thickness of about 0.005 inches, followed by an adhesive promoting layer to a thickness of approximately 0.005 inches, followed by the application of an adhesive layer to a thickness of approximately 0.040 inches achieving a final thickness of approximately 0.050 inches. A primer layer may be applied directly to the outer contact surface of the negative pressure therapy device followed by the application of the adhesive layer directly to the primer to a desired thickness of approximately 0.050 inches. In additional embodiments, an adhesive promoter may be applied to the contact surface of the negative pressure therapy device followed by the application of the adhesive layer to a desired thickness of approximately 0.050 inches.

In certain embodiments, the adhesive layer is a gel layer. As used herein a gel layer is a layer of material that can have properties that are mostly liquid however behave like solids due to the cross-linked nature of its structure. The material chosen for the gel layer may be of a certain cohesive pliable consistency so as to mold to and conform to complex shapes for example imperfections in the skin. As used herein cohesive pliable consistency, elasticity or firmness of the gel layer is defined as the gel layer's ability to flow, mold and stretch and substantially return its original shape when not applied to a surface. The material chosen for the gel layer may also be of a certain tack so as to mechanically secure to the contact area. As used herein tack is defined as the gel's “stickiness” and is the property that allows the immediate formation of a bond on contact with another surface

The adhesive layer material must adhere sufficiently to the therapeutic device such that it stays adhered to the device when the device is removed from the user's skin. Additionally, it must have a tack level that is chosen for appropriate performance at the user's skin interface. That is, at too great a level of tack, removal of the device from the skin can be difficult, painful or injurious. While insufficient tack can allow the device to move during use or allow the seal to the skin to open thereby losing the negative pressure. The level of tack can be measured by a texture analyzer. For example, using a TA.XT plus with a 7 mm radius and 1 inch diameter spherical head the peak adhesion values should be in a range of 200 to 400 grams peak force more preferably 250 to 350 grams peak force and most preferably 275-325 grams peak force.

As discussed above the tack of the adhesive layer is optimized to achieve a releasable but mechanical anchor of the therapy device to the patient. In certain embodiments the contact surface of the flange element is coated with a primer to preferentially anchor the adhesive layer to the negative pressure therapy device over the contact region of the user.

In certain embodiments, the adhesive layer is formed from a washable silicone gel such that when washed and allowed to dry, the adhesive layer returns towards an initial tack. In certain embodiments, the silicone gel is chosen from a group with properties that can be controlled including, but not limited to: cross sectional thickness, degree of crosslinking (and thereby firmness and tack) and viscosity (so as to be processable under desired conditions. As used herein viscosity is measured in cps referring to centipoise (cps) were 1 cps=0.01 g/cm/s.

In an embodiment of the invention, the gel layer is a prepared from a two-part platinum cured organosilicone mixture with properties equivalent to a silicone gel base having an uncatalyzed viscosity of about 30,000 cps and a crosslinker having an uncatalyzed viscosity of about 30,000 cps. The final firmness (cps) of the cured gel may be increased by increasing the proportion of the crosslinker in the mixture or decreased by lowering the proportion of the crosslinker in the mix. The tack of the material can be increased by decreasing the proportion of crosslinker in the mixture or decreased by increasing the proportion of crosslinker in the mix. In order to achieve the desired properties using a silicone gel base of 30,000 cps and a crosslinker of 30,000 cps, the ratio of silicone gel base to crosslinker may range (in parts by weight) from about 10:1 to about 0.5:1, preferably from about 3:1 to about 0.7:1, and more preferably from about 1:1 to about 0.8:1.

In embodiments of the invention, the ratio of 20,000 cps silicone gel base to 300 cps cross linker may further range from about 10.0:0.01 to about 10.0:0.5. In other embodiments of the invention the ratio of 20,000 cps silicone gel base to 300 cps crosslinker may range from about 10.0:0.01 to about 10:0.1. And in further embodiments of the invention the ratio of 20,000 cps silicone gel base to 300 cps crosslinker may range from about 10.0:0.06 to about 10:0.20

By example of the invention the silicone gel base and the crosslinker are mixed in desired ratios and placed under vacuum in order to remove any bubbles in the mixed solution (de-gassing). Following de-gassing, the silicone gel solution is applied to the contact surface of the flange element and allowed to cure. Degassing may not be necessary if gel and crosslinker are prepared in dispense tubes. The mixture can achieve full cure in approximately 24 hours at room temperature however in some embodiments a full cure of the silicone gel may be achieved in about 5 minutes by placing the therapy device containing the silicone gel layer at about 150° C. The cure temperature may be adjusted to suit limiting elements of the therapy device, for example lower melting points of other therapy device elements.

In certain embodiments, the adhesive layer is made of a hydrogel. Hydrogels are a three-dimensional network of crosslinked hydrophilic polymer chains that can be crosslinked either physically or chemically. Due to the hydrogel materials significant water content, hydrogels can resemble natural soft tissue more than any other type of polymeric biomaterial. In further embodiments the hydrogel layer may be found as a hydrocolloid wherein the colloid particles are hydrophilic polymers dispersed in water.

In certain embodiments the adhesive layer is made of a combination of materials applied side-by side on the outer contact surface of the fluidly sealed chamber element. By way of example, a hydrogel material may be applied to the circumference of the center portion of the outer contact surface of the fluidly sealed chamber element and a silicone gel material may be applied on either side peripheral to the hydrogel material. In further embodiments where a combination of materials are applied side-by-side on the outer contact surface of the flange element, a silicone gel layer may be applied to the circumference of the center portion of the out contact surface of the fluidly sealed chamber element and a hydrogel material may be applied to either side peripheral to the silicone gel material followed by a final application of a silicone gel material peripheral to the hydrogel material.

As used herein, “user compliance” refers to the patient's adherence to the prescribed usage of a therapy device for example the usage of a device throughout a sleep cycle.

As used herein, “device compliance” refers to the ability of the device or elements of the device to accommodate variation, for example, bending, twisting, compressing and or expanding of the device in response to device application and usage including anatomical variations of the patient.

Aspects of the device may be made of a generally rigid material. The term “generally rigid” as used herein refers to a material which is sufficiently rigid to maintain the integrity of the particular element in question. The skilled artisan will understand that a number of polymers may be used including thermoplastics, some thermosets, and elastomers. Thermoplastic materials become flowing liquids when heated and solids when cooled, they are often capable of undergoing multiple heating/cooling cycles without losing mechanical properties. Thermoset materials are made of prepolymers which upon reaction cure irreversibly into a solid polymer network. Elastomers are viscoelastic materials which exhibit both elastic and viscous properties and can be either a thermoplastic or thermoset. Common thermoplastics include PMMA, cyclic olefin copolymer, ethylene vinyl acetate, polyacrylate, polyaryletherketone, polybutadiene, polycarbonate, polyester, polyetherimide, polysulfone, nylon, polyethylene, and polystyrene. Common thermosets include polyesters, polyurethanes, duroplast, epoxy resins, and polyimides. This list is not meant to be limiting. Functional filler materials such as talc and carbon fibers can be included for purposes of improving stiffness, working temperatures, and part shrinkage.

Aspects of the device may be formed using a number of methods known to those of skill in the art, including but not limited to injection molding, liquid injection molding machining, etching, 3D printing, etc. In preferred embodiments, the test device base is liquid injection molded, a process for forming thermoplastic and thermoset materials into molded products of intricate shapes, at high production rates and with good dimensional accuracy. The process typically involves the injection, under high pressure, of a metered quantity of heated and plasticized material into a relatively cool mold—in which the plastic material solidifies. Resin pellets are fed through a heated screw and barrel under high pressure. The liquefied material moves through a runner system and into the mold. The cavity of the mold determines the external shape of the product while the core shapes the interior. When the material enters the chilled cavities, it starts to re-plasticize and return to a solid state and the configuration of the finished part. The machine then ejects the finished parts or products.

Those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Structural embodiments of the apparatus may vary based on the size of the device and the description provided herein is a guide to the functional aspects and means.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims:

1. A self-supporting flexible chamber structure adapted for the administration of negative pressure upon the external surface of an individual comprising:
a) a pressure vessel in the form of an approximately spherocylindrical segment comprising a flexible central region of the pressure vessel defining an arch of a height, chordal span, thickness and composed of a material selected to provide minimal bending stiffness such that the internal volume enclosed by the pressure vessel is maintained under negative pressure over a range of neck angles,
wherein an arch section comprises a height to ½ chordal span length ratio of approximately 0.65-0.85
wherein a vertical load vector (FAy) is the pressure multiplied by the ½ the chamber span,
FAy=P*S,
wherein the vertical load vector is the load vector as measured from the apex of the chamber element toward the chordal span,
wherein a horizontal load vector (FAx), is the total load vector (FBx) minus the sum of the negative pressure within the chamber (P), multiplied by the chamber height (H),
FAx=FBx−(P*H)
wherein the total load vector (FBx) is defined by the equation:
FBx=P((S²)+(H²))/2H
wherein β is the negative pressure within the chamber, S is the length of ½ the chordal span of the chamber and H is the height of the chamber measured from the top of the arch to the chordal span line,
wherein at the point of contact of the chamber to the skin of the individual, the total load vector angle (α) is approximately between 65 and 79 degrees, a neck to chordal span line angle (β) is approximately between −18 and −24 degrees, the ratio of the total load vector (FBx) to the translated outward load vector ranges from 0 to −0.3 and the inward deflection of the arch is between 0 and 0.4 inches, wherein the total load vector angle (α) is defined by the equation:
α=arctan (FAy/FAx)
wherein the translated lateral load vector (Fal) is defined by the equation:
Fal=FBx*COS(α−β),
wherein β is the angle of the neck with respect to the chordal span line;
b) an aperture through the self-supporting flexible chamber element; and
c) an air pump operably connected to the chamber element through the aperture to produce a therapeutic level of negative pressure within the chamber element.

2. The self-supporting flexible chamber structure of claim 1, wherein
the spring point of the arch is the contact surface of the chamber element to the skin of the individual and is adapted to form a sealing surface when mated to the individual, and
the contact surface of the chamber element is configured to approximately conform to a continuous contact area on the individual defined by a first location corresponding to a first gonion on one side of the individual's mandibular body, a second location corresponding to the individual's mental protuberance, a third location corresponding to a second gonion on the opposite side of the individual's mandibular body, and a fourth location corresponding to the individual's thyroid cartilage.

3. The self-supporting flexible chamber of claim 1, wherein
an arch section with a height of approximately 1.02 inches, a ½ span length of approximately 1.39 inches, a thickness of approximately 0.17 inches, a material composition of Shore A durometer 40 silicon rubber providing an approximate chamber stiffness of 0.146 N/mm, under an approximate negative pressure of 0.43 psi, is applied to external surface of an individual,
the total load vector angle is approximately 72 degrees, and
at the point of contact of the chamber to the skin of the individual, a neck angle of approximately between −18 and −24 degrees is present, the ratio of the total load vector to the translated outward load vector ranges from 0 to −0.3 and the inward deflection of the arch is between 0 and 0.4 inches.

4. The self-supporting flexible chamber of claim 2, wherein
an arch section with a height of approximately 1.02 inches, a ½ span length of approximately 1.39 inches, a thickness of approximately 0.17 inches, a material composition of Shore A durometer 40 silicon rubber providing an approximate chamber stiffness of 0.146 N/mm, under an approximate negative pressure of 0.43 psi, is applied to external surface of an individual,
the total load vector angle is approximately 72 degrees, and at the point of contact of the chamber to the skin of the individual, a neck angle of approximately between −18 and −24 degrees is present, the ratio of the total load vector to the translated outward load vector ranges from 0 to −0.3 and the inward deflection of the arch is between 0 and 0.4 inches.
